(19) United States
(12) Patent Application Publication
(10) Pub. No.: US 2018/0235914 A1
(43) Pub. Date: Aug. 23, 2018

(54) PROTOTYPICAL MOLECULAR PHARMACOLOGIC PROFILE OF LIPOPHILIC GABAERGIC A & B AGONISTS AGAINST NEURODEGENERATIVE AND NEURODEVELOPMENTAL DISEASES

(71) Applicant: Alexandre Vamvakides, Ymittos Attikis (GR)

(72) Inventor: Alexandre Vamvakides, Ymittos Attikis (GR)

(21) Appl. No.: 15/440,952

(22) Filed: Feb. 23, 2017

(51) Int. Cl. A61K 31/19; A61K 31/165

(52) U.S. Cl. CPC A61K 31/19; A61K 31/165

(57) ABSTRACT

The present invention concerns the prototypical pharmacologic profile of GABA-stearamide, GABA-palmitamide, GABA-linoleamide and Progabide characterized by their interesting positive modulation of at least one γ-aminobutyric acid A receptor being GABA (A) and GABA (B), which are principally extrasynaptic, for treating a neurodegenerative or neurodevelopmental disease. The present invention further originating brain pro-GABAergic and anti-glutamatergic activities and also an unique pharmacological profile which antagonize all the components of the vicious circle generated in the brain by the Glutamate/GABA imbalance, with protective and preventive action against all the behavioral abnormalities, the cognitive deficits and neurotoxicities, in neurodegenerative (Alzheimer's, Parkinson's Huntington's and Multiple Sclerosis) diseases and neurodevelopmental diseases (Autism spectrum desorders and related syndroms).

### BACKGROUND OF THE INVENTION

### Field Of The Invention

The present invention concerns the prototypical pharmacological profile and the new putative brain preventive properties, against neurodegenerative and neurodevelopmental diseases, of the highly lipophilic molecules GABAstearamide (GS), GABApalmitamide (GP), GABAlinoleamide (GL) and (4-([(4-Chlorophenyl) (5-fluoro-2-hydroxyphenyl)-methylene]amino) butanamide: Progabide (PG) [1-2], which were characterized by extrasynaptic agonisms on the γ-aminobutyric acid (GABA) A and GABA (B) receptors.

Of particular interest is the above positive modulation of the GABA (B) receptors, exercing a corrective presynaptic inhibition of the brain glutamatergic pathways and GABAergic interneurons, which exhibited high aberrant activation in neurodegenerative diseases, principally in Alzheimer's disease (AD) but also in Huntington's chorea (HC), Parkinson's disease (PD), or Multiple Sclerosis (MS) as well as in neurodevelopmental diseases like Autism Spectrum Disorders (ASDs), in which the aberrant glutamatergic activation induced seizures (usually without convulsions) and GABAergic interictal activity (mostly via GABAergic interneurons) originating cognitive deficits [3] and various pathological profiles (AD, HC, PD, MS and ASDs), according to the brain regions (Basal Ganglia, Cortex, Hippocampus) affected by this aberrant activations, characterized by seizures and progressive neurodegenerations.

Moreover, GS, GP, GL and PG, by their above GABA(B) negative modulation of the brain glutamatergic pathways , exhibited an efficient control against the brain cholinergic aberrant hyperactivation and the resulting altered theta brain oscillations and cognitive deficits [4].

The present invention relates to a prototypical profile of GS, GP, GL and PG, for use in connection with at least preventive activity against the neurodegenerative and neurodevelopmental diseases.

### DETAILED DESCRIPTION OF THE INVENTION

The present invention concerns the prototypical profile and the putative preventive properties of the lipophilic ligands of GABA(A) & (B) extrasynaptic receptors: GS, GP, GL and PG, against the neurodegenerative or neurodevelopmental diseases.

Indeed, the author discovered that these compounds exhibited a molecular pharmacologic profile, with putative preventive and protective properties against the pathophysiological components of the above diseases, characterized by similar brain neurodegenerative phenomena appearing, respectively, in the late or early stades of the human life [5]. Indeed, despite a variety in the factors triggering these diseases (genetic, pathological or environmental) their evolution follows similar processes, i.e.:—(1) reinforced glutamatergic activity in the brain and disturbed Glutamate/GABA balance inducing aberrant hyperactivation of the GABAergic interneurons in the striatum, nucleus accumbens and, particularly, in the cortex, with downregulation of the big GABAergic pathways: striato-nigral and pallido-ventral tegmental area (VTA).

Downregulation of the above big GABAergic pathways induce the activation of the ascending dopaminergic pathways to striatum, nucleus accumbens and cortex releasing dopamine (DA), the oxidative metabolism of which, will reduce the redox site of the extrasynaptic glutamatergic NMDA receptors, then activating them [6] and amplyfing the Glutamate/GABA imbalance of the brain.

A forward analysis of the resulting new dynamics (in the brain glutamate, GABA, cholinergic and DA pathways) triggered by the Glutamate/GABA imbalance and also the aberrant hyperactivation of the GABA interneurons, make it easy to conclude that,—(2) the initiating factors (soluble Aβ, anoxia, autoimmune reactions, toxics) trigger a vicious circle, which will be continuated with the NMDA induced aberrant activation of the cholinergic neurons releasing acetylcholine (Ach), which, by its action on the astroglial nicotinic receptors, will release glutamate [7], amplify the Glutamate/GABA imbalance and initiate the neurodegenerative processes [5, 7]. Moreover,—(3) the vicious circle will be aggravated by the extrasynaptic glutamatergic and cholinergic stimulations of the GABAergic interneurons in the striatum, nucleus accumbens and cortex, which will amplify the downregulation of the striato-nigral and accumbal-VTA GABAergic pathways with further disinhibition of the ascending DA pathways (nigrostriatal, VTA-nucleus accumbens and VTA-cortex) potentiating the NMDA receptors and the aberrant glutamatergic activity in striatum, nucleus accumbens and cortex, on the one hand, and, on the other, amplifying the inhibitory effects of the nigral GABAergic projections to thalamus and reticular formation which will allow the propagation of the aberrant synchronous cortical glutamatergic hyperactivity [8] and the frequently observed seizures, usually without convulsions but accompanied by cognitive deficits [3, 4], in the neurodegenerative and neurodevelopmental diseases.

It is, therefore, evident that all the described negative behavioral and cognitive consequences, obtained in the appropriate in vivo animal models of the above diseases, derived from the described vicious circle, i.e., following genetic or toxic environmental factors, autoimmune reactions and/or hypoxia, the glutamatergic (NMDA) activation [9], in the neonatal stage of life could initiate the above vicious step (1) and anxiety, fear, social disconnection, compulsive reactions, agressivity and Glutamate induced, stereotypies [9], when amplified in step (2), and seizures, lethargy and cognitive deficits, with step (3), in neurodevelopmental diseases and ASD.

In neurodegenerative diseases and, in particular AD, soluble Aβ, GRK5 deficits and/or hypoxia [10] induce step (1). However, step (2) of the vicious circle is usually expressed by, DA induced, delirant or psychotic symptoms. In PD and HC, the amplified aberrant glutamatergic activity in steps (2/3) caused, respectively, tremor or dyskinesias (motor and orofacial) induced by the neurotoxic aberrant glutamatergic hyperactivity on, respectively, striatal DA or GABA neurons, while in MS it caused moving disturbances, anxiety, unstable mood, depression, seizures [11] cognitive deficits and neuropathic pain [12].

Concerning the neuropathic pain, there is presently evidence that it is a frequent manifestation of all the neuredegenerative diseases [13].

GS, GP, GL and PG via their pharmacological properties could antagonise all the components and steps of the above described vicious circle. Firstly, by their GABA (B) induced negative presynaptic modulation of glutamatergic neurons, they could inhibit the activation of the GABAergic and cholinergic interneurons and disinhibit the striatonigral and pallido-VTA big

GABAergic pathways, annulling the aberrant activation of the ascending dopaminergic pathways, inhibiting the excessive DA release and the NMDA receptors potentiation. Moreover, the above positive modulation of the GABA (B) receptors, situated presynaptically on the glutamatergic neurons, train an inhibitory action against the aberrant glutamatergic activation particularly in the cortex [14]. In addition, GS, GP, GL and PG, by their agonism on GABA (A) and GABA (B) receptors, develop an efficient extrasynaptic dendritosomatic nigral inhibition of the efferent GABAergic pathways projecting to thalamus and reticular formation, disinhibiting and disynchronising the latter and, therefore, arresting the propagation of the hypersynchronous glutamatergic cortical hyperactivity and the induced seizures [8].

Finally, GS, GP, GL and PG, by inhibition of the aberrant hyperactivation of GABAergic interneurons and also those of cholinergic interneurons and cortical pathways could prevent the cognitive deficits and neurodegenerations [3, 4].

In good agreement with the above pharmacological properties, GS, GP, GL and PG, at doses of 50 to 200 mg/kg, intraperitoneal (ip), in mice antagonized all the behavioral effects (in mice) corresponding to the symptoms, in humans, announcing the development of the neurodegenerative and neurodevelopmental diseases, i.e., anxiety, fear, social disconnection, agressivity, compulsive reactions, hypermotility, stereotypic activity, pain, seizures and lethargy, appearing at various degrees according to the amplitude of the Glutamate/GABA imbalance and to the brain regions (Basal Ganglia, Hippocampus, Cortex) affected by the above imbalance. Indeed, all the above behavioral effects (obtained, in mice, by administration of the GABAergic antagonist Pentylenetetrazole (PTZ) at 10 to 70 mg/kg, (ip): with 10 to 40 mg/kg (ip) giving fear, anxiety, social disconnection, and agressivity, hypermotility and stereotypic activity—the latter being better expressed by previous (reserpine induced) hypersensibilisation of the brain DA receptors—and seizures (obtained at 50 to 70 mg/kg (ip) of PTZ), were antagonized by GS, GP, GL, and PG at 50 to 200 mg/kg (ip).

Moreover, their GABA(B) presynaptic inhibitory control on the aberrant hyperactivation of the glutamatergic pathways and cholinergic interneurons constitute an efficient negative control of the cholinergic interneurons and allow protection against the altered theta brain oscillations and cognitive deficits [4], resulting from the above described vicious circle.

However, all these GABAmimetics have weak effects against PTZ or MES induced clonic and tonic crises in mice [15, 16], clearly indicating their highly interesting GABA(B) presynaptic inhibitory activities, on the brain glutamatergic pathways and on the GABA interneurons, which control, respectively, their aberrant hyperactivation and the resulting generation of interictal spikes and cognitive deficits [3].

It is, therefore, evident that GS, GP, GL and PG could not be envisaged as performant antiepileptic drugs, because, their above described beneficial GABA (B) presynaptic inhibitory controls, limited also the hyperactivation of the big GABAergic nigrostriatal neurons, which play a determinant role for the obtention of an performant antiepileptic activity [8].

However, these GABAmimetics increased significantly the latency of PTZ clonic/tonic convulsions and of the lethality, after the PTZ fatal tonic crises, when administered at 50 to 200 mg/kg (ip) in mice, clearly indicating their protective effect against the, brain seizures induced, mitochondrial toxicity, which, chronically, lead to neurotoxicity and neuronal apoptosis, in the neurodevelopmental and neurodegenerative diseases.

Further analysis of the experimental results obtained with GS, GP, GL and PG, in the appropriate animal models (i.e. haloperidol or resepine induced catalepsy, reserpine palpebral ptosis, motor or oral stereotypies by apomorphine and oral stereotypies by haloperidol, in Rats), suggested a more beneficial action of GS and PG (preferentially acting in substantia nigra) in the prevention and neuroprotection against AD, MS and ASDs and relative syndroms. In contrast, the preferentially acting in the striatum, GL and GP, could be more beneficial against HD et PD.

In conclusion, GS, GP, GL and PG, by their above GABA(A) and (B) agonisms, could develop a preventive efficient opposition against the initiation of step (1), and the amplification step (2), of the brain neuropathological processus (glutamatergic (NMDA) and interneuronal GABAergic and cholinergic aberrant hyperactivities), protecting against the step (3), (non convulsive) seizures induced, cognitive deficits and neurodegenerative evolution of neurodevelopmental and neurodegenerative diseases. This preventive action of the above GABAergics must not be confused with the past clinical trials of Progabide on spasticity or tardive dyskinesias symptoms.

Indeed, it is noteworthy, that the brain tonic GABAergic A and B activities are exerted exclusively via extrasynaptic GABA A and B receptors and, therefore, GS, GP, GL and PG by their low affinities, for the GABA receptors, and their high lipophilicities (more specifically for GS, GP and GL) exerted selective extrasynaptic GABAergic A and B activities. In contrast to the higher GABA receptors affinities drugs, used clinically, without success (in particular against the neurodevelopmental diseases), GS, GP, GL and PG constitute the most appropriate ligands against the aberrant glutamatergic, cholinergic and interneuronal GABAergic brain hyperactivities observed in the neurodegenerative and neurodevelopmental diseases.

### REFERENCES

1. Worms P. et al., J Pharmacol Exp Ther., 1982, 220 (3): 660-671.
2. Kolocouris N. et al., Ann Pharm Fr., 1985, 43 (4): 389-396.
3. Feldt Muldoon S. et al., Brain, 2015, 138 : 2875-90 and Bezzina C. et al., Plos One, 2015, PMID 25768013.
4. Siwek M. E. et al., Neural Plast., 2015, PMID 25922768.
5. Kern J. K. et al., Transl Neurodegener., 2013, PMID 23925007.
6. Levy D. I. et al., Neurosci Lett., 1990, 110 (3): 291-6 and Vamvakides A., Ann Pharm Fr, 1992, 50 (5-6): 277-89.
7. Talantova M. et al., PNAS, 2013, E2518-E2527.
8. Iadarola M. J. and Gale K., Science 1982, 218: 1237-40.
9. Frye R. E. et al., Front Neurosci, 2016, PMID 27242398 and Aida T. et al., Neuropsychopharmacology, 2015, 40 (7): 1569-1579.
10. Singh P. et al., Behav Brain Res, 2016, 302: 29-34.
11. Marrie R. A. et al., Mult Scler, 2015, 21 (3): 342-9 and Nicholas R. et al., Mult Scler, 2016, 22 (1): 25-35.
12. Potter L. E. et al., J Neuroinflammation, 2016, PMID 27282914.
13. Reichling D. B. and Levine J. D., Ann Neurol, 2011, 69 (1) : 13-21 and Polli A. et al., Mov Disord, 2016, PMID 27704616
14. Vizi E. S. et al., Br J Pharmacol, 2010,160, 785 -809.
15. Locher W., Arch Int Pharmacodyn Ther, 1985, 276 (2) : 263-70.
16. Vamvakides A., J Pharmacol (Paris), 1986, 17 (3): 323-327

What is claimed as being new and desired to be protected by Letters Patent of the United States is as follows:

1. A compound comprising an effective amount of a compound selected from the group of GABAstearamide (GS), GABApalmitamide (GP) , GABAlinoleamide (GL) and Progabide (PG), said compounds being agonists of brain y-aminobutyric acid (A) [GABA (A) (+)] and [GABA (B) (+)] receptors (principally extrasynaptic), with brain protective and preventive activities against neurodegenerative and neurodevelopmental diseases.

2. A pharmaceutical composition comprising an effective amount (30 to 300 mgs, per os, daily) of a compound at least one pharmaceutically acceptable excipient said compound being selected from the group consisting of GS, GP, GL and PG, and combination thereof, said compound being [GABA(B)(+)] and [GABA(A)(+)], with brain protective and preventive activities against neurodegenerative and neurodevelopmental diseases.

3. The method according to the claim 2 wherein said pharmaceuticals with brain protective and preventive activities against neurodegenerative and neurodevelopmental diseases of brain condition were selected from the group consisting of Alzheimer's disease, Huntington's disease, Parkinson's disease, Multiple Sclerosis, Autism spectrum disorders and also all the brain neurodegenerative or neurodevelopmental syndroms originated from, respectively, late or early stage human life, brain Glutamatergic/GABA imbalance.
